(19) United States
(12) Patent Application Publication
(10) Pub. No.: US 2026/0000363 A1
(43) Pub. Date: Jan. 1, 2026

(54) Protable device and method for non-invasive measurement of the level of physiological values sPortable device and method for non-invasive estimation of the level of

(71) Applicant: SMARTHEALTH, S.L., Jàvea (Alicante) (ES)

(72) Inventor: Salvador Albalat Estela, Valencia (ES)

(21) Appl. No.: 18/831,143

(22) PCT Filed: Feb. 8, 2023
(86) PCT No.: PCT/ES2023/000002

§ 371 Date: Feb. 19, 2025

(30) Foreign Application Priority Data
Feb. 11, 2023 (ES) P202230112

(51) Int. Cl. A61B 5/00; A61B 5/0205; A61B 5/024; A61B 5/0531; A61B 5/0537; A61B 5/145; A61B 5/1486

(52) U.S. Cl. CPC A61B 5/7267; A61B 5/0017; A61B 5/02055; A61B 5/02416; A61B 5/02438; A61B 5/0531; A61B 5/0537; A61B 5/14532; A61B 5/1486; A61B 5/681; A61B 5/6897; A61B 5/7435; A61B 2562/0238

(57) ABSTRACT

A portable device and method for non-invasive estimation of the level of physiological values such as blood glucose and blood cholesterol, comprising a central processing unit (1) which includes connections to: a signal emitter which emits signals via electrodes in contact with the skin, which, when processed by a bioimpedance microcontroller and said central processing unit (1), provide values such as hydration, body mass index, bone index; a digital optical sensor (4) which allows calculation of the blood oxygen value, heart rate and temperature; and enzymatic sensors (5) which determine the quantity of glucose or lactate; said central processing unit (1) calculating the physiological values on the basis of an automatic learning algorithm that has been trained with a set of clinical history data from a group of patients for whom at least values of bioimpedance, temperature, oxygen and heart rate are available.

### SECTOR OF THE INVENTION

The field of the invention described herein falls within the area of research or analysis of materials by determination of their chemical or physical properties (measurement, research or analysis procedures other than immunological assays, in which involving enzymes or microorganisms). In the investigation or analysis of materials by the use of optical means, i.e. using infrared, visible or ultraviolet rays. Measures aimed at establishing a diagnosis

More specifically, the subject matter of the invention has a place in biomedical engineering and the medical technology, for the development of portable electronic devices for monitoring physiological variables of people and their state of health, in general, and blood glucose and cholesterol levels, in particular.

### BACKGROUND OF THE INVENTION

Worldwide, 425 million people have diabetes mellitus and it is estimated that this figure will increase to 629 million by 2045 as a result of population growth and aging, increasing urbanization, the prevalence of obesity, sedentary lifestyles and other unhealthy lifestyles. One in eleven adults has diabetes and one in seven pregnancies is affected by gestational diabetes. Efficient control of the disease requires monitoring of the blood glucose level. Glucometers, which measure the glucose level from blood samples, are most commonly used devices for glucose measurement because of their accuracy. This method is painful and inconvenient, especially in cases where it is necessary to glucose level monitoring. To avoid this problem, numerous methods for the noninvasive measurement of blood glucose have been proposed in recent years.

Impedance spectroscopy is based on the injection of current at multiple frequencies and the measurement of the voltage produced in the body region of measurement. The measurement of glucose is performed indirectly by analyzing its influence on the impedance spectrum. Some examples of patents based on this technique are ES2445700, ES2582185, WO2007/053963, US2005/0192488, US2016/0007891 and US2015/016438.

The bioimpedance measurement technique is based on the injection into the human body or tissue to be measured of an alternating electric current of very low intensity, well below the thresholds of perception. The electrical current produces a drop in electrical voltage, the greater the electrical impedance of the tissue.

This technique began to be applied in 1930, when Atzler and Lehman found that fluid changes in the thoracic cavity as a result of blood pumping by the heart also produced changes in thoracic impedance. Holzer et all were the first to apply an alternating signal to avoid electrode polarization problems in bioimpedance measurements. In 1966, in collaboration with the Apollo program, the first device for monitoring hemodynamic parameters was developed, paving the way for the development of impedance cardiography for systolic volume estimation. This principle is the basis of the impedance cardiograph, which has also been used for several decades for the estimation of cardiac output by means of the equation by Kubicek.

Since then, bioimpedance has been applied in the development of new medical diagnostic instruments and devices. In 1978 Webster and Henderson attempted to reproduce X-ray tomography techniques by applying low-frequency electrical signals to the frequency. But it was not until the 1980s, when the University of Sheffield developed the basis of what is now known as electrical impedance tomography, from which, by measuring the electrical potentials on the surface of the body, images can be obtained related to the distribution of impedances inside a body.

Taking into account that tissue impedance changes according to the physiological state of tissues, this technique has also been used to monitor the viability of transplanted organs, to determine the state of hydration of the skin or the diagnosis of skin pathologies, and even as a method for noninvasive measurement of blood glucose levels. Bioimpedance has also been used in the clinical laboratory. as a tool for cellular measurements (cou/ter counter, hematocrit measurements or cell culture monitoring) and the detection of substances in Lab-on-a-Chip.

One of the most important applications of bioimpedance is the study of the body composition, of great clinical utility in different areas: nephrology, nutrition, obstetrics, gastroenterology, in postoperative follow-up, in patients infected with HIV, with growth hormone deficiency, obese or in critical care. In 1963, Tomasset made the first estimates of total body water from whole-body bioimpedance using a fixed-frequency alternating current.

Since then, bioimpedance measurements have been used extensively in numerous patents, which present methods and devices both for the quantification of body composition, estimation of fluid volumes and anatomical localization of masses (muscle, fat, water), as well as for other applications such as estimation of blood pressure, systolic volume, cardiac output, respiratory rate and heart rate, blood glucose level or tissue monitoring, among others.

There are several patents that explain the internal components that make up the devices that protect, as well as their operation, for obtaining the signal of bioimpedance. Those patents that show greater detail are limited to describing a global overview of the main elements used by the patented device to carry out the measurement. In general terms, the scheme employed comprises the following elements: a sensing stage composed of several electrodes together with the electronics responsible for capturing the bioimpedance signal, which normally includes filtering stages, amplifiers, and analog-to-digital (A/D) and digital-to-analog (D/A) converters; a processor or computing element; a memory for storing relevant data; and, in some cases only, a communications stage for sending the processed data to the outside. However, the degree of description at the internal level of these modules is usually insufficient, and in particular, the analysis of the detection and signal conditioning electronics is mostly scarce.

The patent (U.S. Pat. No. 7,917,202), whose main contribution to the state of the art is a refined model that includes contributions from intracellular tissues to allow more accurate measurement at two or more frequencies. However, for the measurement of the signal from the bioimpedance, the authors refer to specialized medical instrumentation (from the manufacturer Xitron Technologies), without going into further details.

The patent (U.S. Pat. No. 6,615,077), which includes a method for determining the body dry weight of a patient using segmental measurements based on electrical bioimpedance analysis, also uses a solution from the same manufacturer for bioimpedance data collection. Again, the patent (U.S. Pat. No. 7,945,317), which describes an improved multifrequency method for performing a bioimpedance analysis of a subject's body segment, suggests that a commercial solution be used for current application and voltage recording. Lo is the same for the patent (US20110275922), which in this case states that data processing can be performed using this equipment or on-line using a separate computer. The patent (US20060122540) provides a method for determining the hydration status of patients on peritoneal dialysis and hemodialysis, and describes among the modules employed a device for continuously calculating the circumference of a body segment, based on a digital signal processor (DSP). However, like the previous ones, it is recommended to use medical instrumentation from the referenced manufacturer for the measurement system.

On the other, there is a set of patents that make specific contributions to the scheme. global previously raised, for the improvement of some of the elements of the electronics of the device involved in the measurement process. s. For example, the patent (US20130030046165), describing a capacitive bioimpedance sensor, which includes an ad-hoc signal pre-processor, which is coupled to the sensor. Further, in one preferred embodiment, the sensing circuitry measures relative impedance by employing one or more Wheatstone bridges. The patent (US20060004300) presents a method for estimating bioimpedance at multiple frequencies using a Linear Feedback Shift Register (LFSR) circuit, which produces a pseudo-random sequence that feeds the D/A converter.

The patent (U.S. Pat. No. 7,457,660) includes a mechanism for eliminating errors in bioimpedance measurements, based on the separation of the bioimpedance value from other sources of error from measurements on two similar body sections. The patent (US20050012414) presents an apparatus specially designed to provide a second power supply for electronic devices of the floating type, and thus is ensures that the device meets the medical safety requirements for bioimpedance measurement.

The patent (US20040171963) presents a method for a more accurate estimation of the body composition that corrects a bioelectrical impedance parameter, which reduces the load on the extracellular fluid distribution. It employs an electrode exchange unit that allows the use of configurations of up to 8 electrodes.

In the patent (U.S. Pat. No. 7,974,691) they refer to Hartley et al. measurements 20 microsecond microampere pulses repeated at 50 millisecond intervals in which the voltage response is measured. The impedance circuit of the patent (U.S. Pat. No. 6,370,424) uses a balanced two-phase current pulse that prevents net charge transfer to the electrodes, thus reducing electrode corrosion and deposition for improved biocompatibility. The patent (US20100081960) presents a bioimpedance sensor that stands out, compared to other approaches based current injection into the tissue, for using optical methods whose precision is greater, although the associated electronics are also more accurate. The patent (EP2567657A1), in one of its claims, includes as its main contribution the grouping of a reference unit and one or more measurement units connected together on a bus that includes one or more electrical conductors. On the other hand, the patent (US20070142733) presents a method of signal separation based on a specific algorithm that is performed in part in an implantable device, in order to reduce interference.

The patent (U.S. Pat. No. 7,706,872) describes a method for measuring electrical bioimpedance. characterized by a periodic excitation signal in the form of square pulses, which is applied to the input of the object to be measured, whose output is connected to a synchronous detector. The patent highlights that the use of rectangular signals ensures that the device has a simple design and low power consumption, and describes a method for increasing the accuracy of bioimpedance measurements by means of a set of functional blocks.

Although some of them have been mentioned, there is a broader set of relevant features to be included in the design of patented bioimpedance devices, such as their portability, low cost, low energy consumption, ability to communicate with the environment, and user customization, among others. This opens the way, through the use of of these devices, to the development of new emerging healthcare paradigms, such as e-Health or m-Health.

The patent (U.S. Pat. No. 7,930,021) details a small-sized apparatus for the measurement of the body composition, by means of electrodes placed on the handle of the device, which must be held by both hands. The advantage of this device over others is its size, which allows it to be carried by the subject. In one of the claims of the patent (US20050101875), which is generally intended for monitoring cardiac vital signs, a monitor/sensor is presented that is preferably portable, of low cost and limited battery, which may be disposable. In addition, the monitor electronics may include a wired or wireless link to transmit data.

The patent (US20130030046165) discussed above also features a low-cost disposable capacitive sensor. The patent (U.S. Pat. No. 6,532,384) features a device portable device with buttons and a display. Patent (U.S. Pat. No. 7,783,344) in one of its implementations, includes measurement of segmental impedance, with wireless transmission capability to a remote device. Patent (U.S. Pat. No. 5,876,353) features an impedance monitor for detecting edema through respiratory rate assessment, which communicates wirelessly with a wrist-worn device and the wrist-worn device communicates wirelessly with a remote device. remote fixed device over the telephone line. Another complementary approach is to use the processing capacity and connectivity of commercial portable devices, such as a PDA (U.S. Pat. No. 6,790,178), to process multiple physiological variables (including bioimpedance). In this case, the sensors are coupled to the PDA or have the possibility of transferring the data to a memory that can then be inserted into the PDA. In the patent (US20120035432), an interesting device is discussed that can communicate with the healthcare provider within the same room or remotely wirelessly via an intermediate device, establishing a bidirectional communications system.

On the other hand, the document (US20120035432) raises another relevant design issue: customization in bioimpedance measurements for the specific characteristics of a patient. However, no patent has the ability to adapt in real time to the user without their intervention.

Document (ES2774983) describes a portable device for the non-invasive estimation of blood glucose level, comprising a measuring unit and the personal monitoring unit, communicating with each other wirelessly. The measuring unit is a portable device that is placed on the skin of an area of the human body irrigated by a vascular bed, and which emits light at two different wavelengths, one of which corresponds to an absorbance maximum in the absorption spectrum in the glucose molecule within near-infrared range. The measuring also captures the light passing through the measurement area, and the personal monitoring unit estimates the blood glucose level based on this information, displaying the result of the estimate to the user.

### DESCRIPTION OF THE INVENTION

The present invention relates to a device for the non-invasive estimation of the level of glucose and other values such as cholesterol or triglycerides in the blood.

With respect to the common devices for estimating arterial glucose levels (glucometers), these require a blood sample in order to be able to perform measurements on test strips. The integration of the system described above in various devices such as a smart watch or a computer mouse allows continuous measurements to be taken without the need to prick oneself each time. In addition, the system allows remote monitoring of the patient by the medical specialist and the use of alarms to changes in glucose levels or other parameters that may affect the patient's health.

Therefore, the main advantages are:
Safe and painless use that avoids any type of discomfort or inconvenience to the user.
Continuous patient monitoring. Measurements can be repeated as many times as desired.
Low cost, since it uses commonly used electronic components and does not require test strips that would increase the ongoing cost of the device.

There are continuous glucose measurement systems based on measuring technologies interstitial fluid. With respect to commercial clinical systems for automatic/semi-automatic monitoring of glucose in interstitial fluid, their main advantages are:
Low cost (it does not need complements that make the continuous cost more expensive),
Harmless (no need for insertion of elements under the skin that can produce irritations, in addition to the danger of infections that this entails),
Accuracy, since it analyzes the blood glucose component itself and not that of the interstitial fluid, which can be misleading.

In addition, these devices have other innovative features and technical advantages:
The measuring principle is based on the effects of various techniques such as bioimpedance, optical and enzymatic sensors, so that the measurements are harmless and can be repeated as many times as desired without disturbing the user.
It is a portable system capable of communicating with the exterior through bidirectional wireless communications, for the integration of measurements in an e-Health system upstream, and remote configuration and customization of the device downstream.
The device object of the invention is based on the technique of bioimpedance spectroscopy for the acquisition of values such as glucose value and the technique of optical sensors for the acquisition of values such as oxygen value or frequency. and the bioenzymatic technique to take complementary glucose or lactate.

Compared to other proposals based on the bioimpedance spectrometry technique and photoplethysmography techniques, the device described in the present invention presents a series of novelties and innovations:
1. Access to the arterial component of the blood by identifying changes in resistance and impedance in the electrical signals emitted and captured by self-developed sensors.
2. Relative normalization to fluctuations in the level of the captured signals by means of electrical or bioimpedance signals through the incorporation of values captured by light, movements, and other conditioning factors, consisting of a comparative analysis with respect to continuity in the captured signals and their correlation between the values by means of automatic learning algorithms.

3. Determination of values and estimation of blood values of physiological parameters such as glucose for each individual based on Machine Learning algorithms that have been developed from the correlation of different values depending on the particular characteristics of the individual and values such as the oxygen, temperature or heart rate among others, which represent the context in which the measurement is made. The novelties of the object of the invention are reflected in the set of claims accompanying this description.

The intelligent system which is proposed at this document possesses a series of functionalities described in the form of novel features that none of the reviewed papers fully meet. The main contribution is the combined use of the bioimpedance technique (which analyzes body composition, indicating the approximate amount of muscle, bone and fat, etc.) with photoplethysmography (a plethysmography technique in which a beam of light is used to determine the volume of an organ) and the use of Machine Learning algorithms to improve the accuracy of the system based on the data provided by both techniques.

System components. The main novelty of this system is the existence of a central processing unit (1) that is capable of obtaining data from three sources such as bioimpedance, optical sensor technology and enzymatic sensors; it is also capable of jointly processing these values to obtain, by means of automatic learning algorithms integrated in this unit, which have been previously developed and registered, a correlation of values that calculates in real time the following physiological values:
Arterial glucose
Cholesterol
Lactate
Triglycerides
Hormones
Potassium
Sodium
Other values that are obtained directly by bioimpedance, such as hydration, body mass index, bone mass index, fat index and others.
Other values obtained by the optical sensors such as heart rate, temperature and electrocardiography.

As already indicated, the device (see FIG. 1) is preferably made up of a central processing unit (1) incorporating connections to:
In one of the connections this unit emits signals through electrodes (2) in contact with the skin and then collects the signals through other sensors (3) placed separately in contact with the skin as well.
These sensors (3) allow multiple signals to be obtained at different frequencies between 1 KHz and 150 Khz. These signals are sent to the central processing unit (1) where they are processed by a bioimpedance microcontroller. This microcontroller processes the signals by extracting the resistance and reactance values, from which multiple values such as hydration, body mass index, bone index, etc. are obtained.
The central processing unit (1) also integrates a digital optical sensor (4) that emits a light signal in different colors. This sensor is in contact with the skin and picks up the emitted signal which allows the blood oxygen value, heart rate and temperature to be calculated.
The central processing unit (1) is also equipped with connections for integrating enzymatic sensors (5). These enzymatic sensors collect sweat or saliva samples and can calculate the amount of glucose or lactate in sweat or saliva.
Finally, the central processing unit (1) has connections to a screen (7) for displaying the data obtained and with an interface (8) for entering the data.
The unit is used to manage the device's options.
This device has a rechargeable battery (6) as a power source.

### DESCRIPTION OF DRAWINGS

To complement the description being made and in order to facilitate the understanding of the characteristics of the invention, this description is accompanied by a set of drawings in which, by way of illustration and not limitation, the following have been included represented the following:

FIG. 1 shows in a functional block diagram the components of the device of the invention.

FIG. 2 shows the basic hardware and software components that make up this device.

FIGS. 3-5 show different functional realizations of such a device.

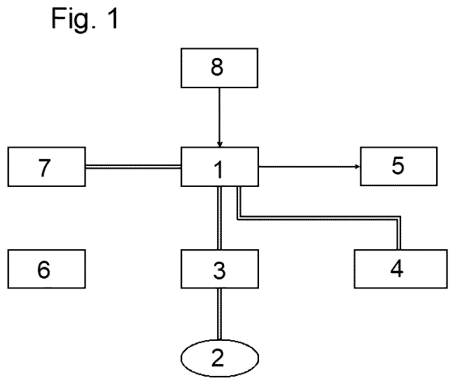

### REALIZATION OF THE INVENTION

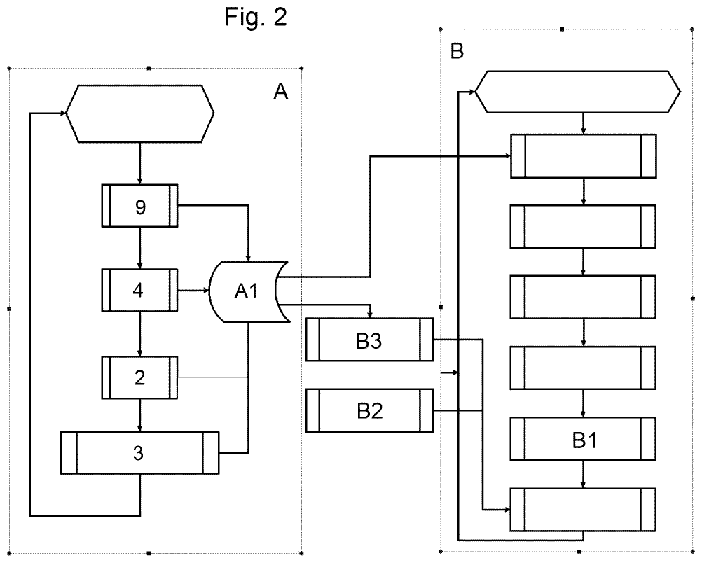

The present invention is based on a system that allows the continuous measurement of physiological values of clinical use such as glucose and cholesterol among many other values and to monitor chronic diseases related to these values. The system, as shown in FIG. 2, consists of a hardware (A) allowing the obtaining multiple data through different types of sensors and a software (B) based on Machine Learning (part of Artificial Intelligence that deals with machine learning) capable of analyzing them and obtaining a high accuracy.

Description of the hardware (A). The main basis of the invention is the use of the bioimpedance spectrometry technology, a technique widely used in hospitals and whose validity has been demonstrated in numerous clinical trials for different uses.

To obtain the bioimpedance data, two sensors (2, 3) are used, one transmitter and one receiver, which emit an electrical signal at 256 different frequencies. The sensors used have been designed by the inventors and as a novelty do not require gel for use and have been validated in this respect.

In addition, the developed hardware integrates digital optical sensors (4) that provide other values such as heart rate and arterial oxygen by means of optical techniques.

The device further comprises a means for measuring temperature (9).

This data (A4) are fed to a Machine Learning algorithm as it allows to adjust the bioimpedance values to the final glucose value prediction result together with those described above for a better accuracy of the set.

The software (B) implemented in this system is one of the main innovations of the system, since it uses artificial intelligence technology, specifically the use of Machine Learning algorithms for the detection of arterial glucose value from the use of impedance spectrometry together with the input of other data to adjust the accuracy of the whole system.

As it is well known, Machine Learning consists of feeding a model with a dataset, train it until it learns a function (or algorithm) that achieves from some input data an output close to the one that had been produced in the sample data with a reasonably high degree of accuracy, even if no similar sample has been analyzed during the training. The dataset with which the model implemented in this device has been fed consists of the historical clinical (B2) of a group of patients for whom bioimpedance, temperature, oxygen, heart rate (B1) values are available. Using a supervised learning support vector machine (SVM), which is commonly used for classification and regression problems, and through a process from the bioimpedance spectrometry value (resistance and reactance) together with the patient's clinical data obtained from their clinical history (B2) and temperature data (B3), an algorithm has been generated to obtain a glucose value with an accuracy of more than 94% of the standard value taken with a conventional glucometer.

The data obtained are represented and sent to a screen (7) of the device for continuously, with the option of continuous or programmed measurement to optimize the device's consumption.

Measurement methodology. Another novelty of the system is the ability to measure directly on the skin on a continuous basis with its own non-fungible sensors and the use and combination of other techniques, such as bioimpedance and optical technology, applying machine learning to measure continuously and accurately. In addition, the incorporation of new patient data on a continuous basis allows the algorithm to be improved on an individualized basis and for the group of users as a whole.

The system is designed to calibrate automatically.

The measurement method is described.
From the beginning of the manual or programmed recording process, the following are issued a current at multiple frequencies.
The magnitude of the phase is calculated.
The system is self-calibrating.
The resistance and reactance are calculated from the magnitude.
data is read with photoplethysmography.
This data is applied to Machine Learning algorithms.
The glucose value is calculated

As we have already mentioned, one of the main innovations of this system compared to those of the existing, in addition to the combination of different sounding techniques, is the use of artificial intelligence to obtain the patient's clinical data such as glucose from the different values obtained by these sensors.

A data acquisition protocol was developed to obtain the algorithm. biomedical data and a study of the data collected so far. With the few data collected, initial prediction models have been made, focusing on relating bioimpedance and glucose data. The data used for the model have been acquired following the established data collection protocol.

With the set of data acquired so far, prediction models have been created. It has been shown in the literature that the glucose level correlates with bioimpedance values; therefore, for the models generated, data on bioimpedance, photoplethysmography and weight and height, taken manually, will be used.

Among the two techniques used to predict a glucose value with the dataset, SVM Gaussian has been used, as it has been found to provide the highest accuracy.

In an improved version, the available dataset has been applied to a deep neural network of up to five layers (Deep Learning) to generate an output algorithm that will generate the value of glucose and others, based on the magnitudes measured by the reading device, with high accuracy.

The device of the invention finds several possible realizations, some of which we will describe below.

A non-invasive electronic micro-device integrated with specific sensors that measure bioimpedance spectrometry and enzymatic sensors both in contact with the skin. together with a digital optical sensor measures physiological values such as glucose, cholesterol, triglycerides, lactate and other values through the combined use of the values provided by the described techniques and the use of machine learning algorithms that combined together allow the optimization of the system and a high accuracy of the same as a medical device for diagnostic utility in chronic diseases through a series of devices described.

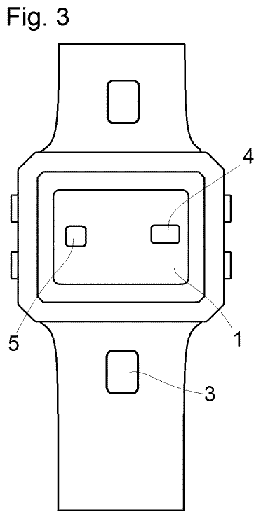

In a preferred embodiment, a smart wristband integrates the components described above into a housing in which the electronic circuitry of the processing unit (1) connected to the bioimpedance sensors (2, 3) is incorporated above. It also has a digital optical sensor (4), preferably a photoplethysmography sensor, and a connection for an enzymatic sensor (5). The device integrates an internal memory with the automatic learning algorithms that are updated and connected to a program via wireless connection. (See FIG. 3).

In another preferred embodiment, the device features the configuration of a smart computer mouse that has two sensors integrated on both sides on which both fingers naturally rest and allows continuous monitoring of health parameters including glucose, hydration, temperature, pulse oximeter, heart rate, and other values.

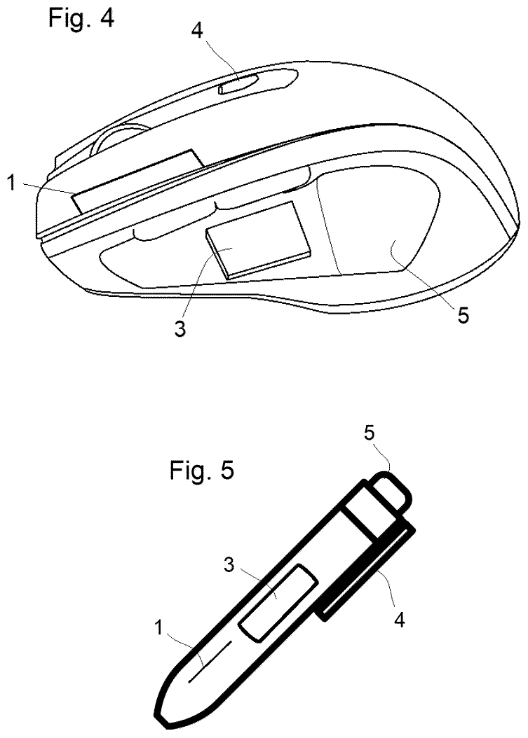

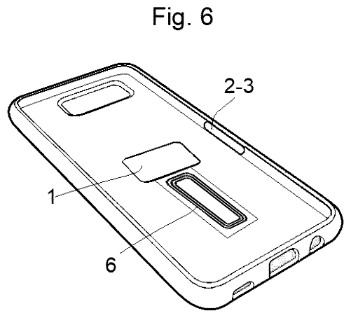

The mouse is powered by the USB cable or by own batteries. Communication is via wireless or USB to the computer (see FIG. 4).

In another alternative embodiment this device presents the conformation of a wireless glucometer, which allows to measure glucose directly through two bioimpedance sensors (2, 3), a digital optical sensor (4) and in the upper part integrates an enzymatic sensor (5). This sensor is individualized and can be interchanged for each user. The innovation is its possibility for portable hospital use and possible home use. The system communicates data to an application or laptop wirelessly.

Another preferred embodiment is shown in FIG. 6, in which it can be seen that it presents a structure of a cell phone case, which is powered through an NFC antenna (6) of the mobile phone itself mobile. This case incorporates bioimpedance sensors (2, 3) on the sides to measure glucose. In this embodiment it is also provided a computer application, which once installed on the cell phone, is able to collect bioimpedance data through the NFC antenna and to determine by means of a machine learning algorithm, implemented in the cloud or in the application itself, the values of glucose, heart rate, blood glucose and heart rate. and oxygen.

1. Portable device for the non-invasive estimation of the level of physiological values, such as the glucose and cholesterol in blood which comprises a unit central de processing (1) incorporating connections to:
an emitter of signals through electrodes in contact with the skin, which are collected through sensors (2, 3) placed separately in contact with the skin, which obtain multiple signals at different frequencies that are processed by a bioimpedance microcontroller that derives from them values of resistance and reactance, on the basis of which the processing unit (1) obtains values for hydration, body mass index and/or bone index;
a digital optical sensor (4) that emits a light signal in different colors, placed in contact with the skin, which collects the emitted signal in order to calculate the value of oxygen in blood, heart rate and temperature;
enzymatic sensors (5), which collect sweat or saliva samples in order to calculate the amount of glucose or lactate in these fluids; and
with a screen (7) for displaying the data obtained and an interface (8) for entering instructions or commands in the unit to manage the device options;
determining said processing unit (1) the estimated physiological values based a machine learning algorithm that has been trained with a set of clinical history data from a group of patients for which at least bioimpedance, temperature, oxygen and heart rate values are available.

2. Device, according to claim 1, characterized in that it presents a structure like a smart bracelet that integrates in a box the electronic circuit of the processing unit (1) connected to some bioimpedance sensors (2, 3), with a digital optical sensor (4), preferably a photoplethysmography sensor, and a connection for an enzymatic sensor (5); as well as an internal memory in which it stores the algorithms of Self-learning that are updated and connected via wireless connection.

3. Device, according to claim 1, characterized in that it has a structure like an intelligent computer mouse that integrates two sensors on both sides on which both fingers rest naturally and allows the continuous monitoring of the fingers. parameters such as glucose, hydration, temperature, oxygen and/or heart rate, which is powered by the USB cable or its own batteries and communicates wirelessly or via USB with a computer.

4. Device, according to claim 1, characterized in that it has a wireless glucometer structure to measure glucose directly through bioimpedance sensors (2, 3), a digital optical sensor (4) that calculates the value of oxygen in blood, heart rate and temperature, and also integrates an enzymatic sensor. (5) individualized for each user, provided with means of data communication obtained wirelessly.

5. Device, according to claim 1, characterized in that it presents a structure of a cell phone case, which is powered through an NFC antenna (6) of the cell phone, and incorporates on the sides of the same bioimpedance sensors (2, 3) to measure the glucose, further comprising a computer application installed on the cell phone, which is able to collect bioimpedance data through said NFC antenna and to determine by means of a Machine Learning algorithm, implemented in the cloud or in the application itself, the values of glucose, heart rate and oxygen.
